(19) United States
(12) Patent Application Publication
(10) Pub. No.: US 2010/0145223 A1
(43) Pub. Date: Jun. 10, 2010

(54) Peripheral Reticular Pigmentary Change and Age-Related Macular Degeneration

(75) Inventors: Eric A. Postel, Durham, NC (US); R. Keith Shuler, Durham, NC (US); Jennifer Vaughn Caldwell, Durham, NC (US); Michael Hauser, Durham, NC (US); Silke Schmidt, Durham, NC (US)

Correspondence Address: BANNER & WITCOFF, LTD., 1100 13th STREET, N.W., SUITE 1200, WASHINGTON, DC, 20005-4051, US

(73) Assignee: DUKE UNIVERSITY, Durham, NC (US)

(21) Appl. No.: 12/617,256

(22) Filed: Nov. 12, 2009

Related U.S. Application Data
(60) Provisional application No. 61/118,269, filed on Nov. 26, 2008.

(51) Int. Cl. A61B 3/14; A61F 9/00; A61B 3/10

(52) U.S. Cl. 600/558; 351/206

(57) ABSTRACT

Examination of 956 age-related macular degeneration cases showed that the complement factor H variant (Y402H, C allele at rs1061170) increases risk for the development of peripheral reticular pigmentary change. AMD phenotypes of 796 carriers of the CFH Y402H variant were compared to the AMD phenotypes of 160 non-carriers. Of 34 phenotypic features analyzed, only peripheral reticular pigmentary change (PRPC) was associated with this CFH variant (P-value 0.0006). The proportion of AMD cases with PRPC correlated with the number of CFH risk C alleles in a dose-response fashion. The association of CFH Y402H polymorphism with PRPC suggests that AMD changes are not limited to the macula. Current AMD grading methods assess only the macula; peripheral retinal changes should also be included in grading methods. PRPC may be used as a surrogate of a high-risk genotype and may be used for diagnostic, therapeutic, and research purposes.

The content of provisional application Ser. No. 61/118,269 filed Nov. 26, 2008, is expressly incorporated herein.

This invention was made using funds from U.S. government grant no. U10 EY12118-05 from the National Institutes of Health (NIH)/National Eye Institute. Therefore the U.S. government retains certain rights in the invention.

### TECHNICAL FIELD OF THE INVENTION

This invention is related to the area of genetic testing, drug discovery, and Age-Related Macular Degeneration. In particular, it relates to phenotypes and genotypes which increase the risk of Age-Related Macular Degeneration.

### BACKGROUND OF THE INVENTION

The leading cause of central vision loss in older Americans is age-related macular degeneration (AMD).¹ The clinical features of AMD are well described but can vary significantly from one individual to another. Most of the degenerative changes from AMD involve the macula; however, more widespread clinical findings have been described including iris color², lens status³ and peripheral retinal changes⁴⁻⁵.

Recent reports demonstrate that a common polymorphism (Y402H, C allele at rs1061170) of the complement factor H gene (CFH) is associated with an increased risk of advanced AMD including both neovascular and geographic atrophy⁶⁻¹¹. CFH is also associated with macular soft drusen⁹ but additional phenotypic characterization is limited.

We have already begun to clarify the phenotype-genotype relationships in AMD.¹¹⁻¹² Further examination may provide clues to the molecular mechanisms and eventually guide treatment recommendations for specific subtypes of AMD. Here we analyze in more detail genotype-phenotype relationships in AMD.

There is a continuing need in the art to identify phenotypic or genotypic markers that are useful for the stratification, prediction of risk, and assignment of appropriate nutritional or medicament regimens.

### SUMMARY OF THE INVENTION

A method is provided for predicting increased risk of Age-Related Macular Degeneration. A subject is examined to determine the presence of peripheral reticular pigmentary change. An increased risk of Age-Related Macular Degeneration is predicted when peripheral reticular pigmentary change is determined in the subject. The increased risk can be used to guide treatment, clinical studies, monitoring, diet, behavior, and environmental exposures.

These and other embodiments of the invention provide the art with additional tools for recognizing and stratifying patients for risk and prevention of neovascular AMD.

### BRIEF DESCRIPTION OF THE DRAWINGS

FIG. 1 Fundus photograph of the inferonasal periphery in the right eye of a patient with age-related macular degeneration exhibits peripheral reticular pigmentary change (PRPC). Normal retina is adjacent to the PRPC in the left side of the photograph. The typical honeycomb-like pattern of PRPC is demonstrated well in this patient.

### DETAILED DESCRIPTION OF THE INVENTION

The inventors have found that the presence of Peripheral Reticular Pigmentary Change (PRPC) in a patient correlates with an increased risk of Age-Related Macular Degeneration. Thus examining a patient to assess PRPC can be used as a means of predicting increased risk.

AMD is typically graded using a number of clinical observations. These phenotypic distinctions are: grade 1 is defined as no drusen or small (<63 um) nonextensive drusen without RPE abnormalities; grade 2 is defined as extensive small drusen or non-extensive intermediate drusen (>63 um, <125 um) and/or retinal pigment epithelium hyper or hypopigmentation, but not geographic atrophy; grade 3 was defined as extensive intermediated drusen or any large, soft drusen (>125 um), including drusenoid retinal pigment epithelial detachment; grade 4 is defined as geographic atrophy (area of RPE atrophy with sharp margins, usually visible choroidal vessels, at least 175 um diameter); and grade 5 is exudative AMD, including nondrusenoid pigment epithelium detachment, CNVM, subretinal hemorrhage or fibrosis, or a photocoagulation scar consistent with treatment of AMD.

Patients that have an increased risk of AMD can be classified in clinical studies as a divided or undivided group. Various risk-reducing behaviors or treatments can be recommended to persons with an increased risk under the guidance of a physician. For example, dietary supplements can be recommended or prescribed to the subject to reduce the risk of progression of AMD and visual loss. Alternatively, a diet may recommended or prescribed to the patient in order to reduce and/or delay onset of symptoms of Age-Related Macular Degeneration. As an alternative, smoking cessation education and/or treatments can be recommended or prescribed for the subject to reduce and/or delay onset of symptoms of Age-Related Macular Degeneration. Control of systemic co-morbidities may halp reduce the risk of advancing AMD. A “watchful waiting” program can alternatively be used in which a monitoring schedule is set up for the subject to monitor onset and/or progress of Age-Related Macular Degeneration on a regular basis, in an effort to “catch” subtle progression early and administer appropriate treatment (e.g., anti-VEGF therapy). Supplemental or dietary anti-oxidants can be recommended or prescribed to the subject to reduce and/or delay symptoms of Age-Related Macular Degeneration. A further option is the recommendation or prescription of a healthier lifestyle, perhaps including exercise to the subject to reduce and/or delay onset of symptoms of Age-Related Macular Degeneration. Anti-inflammatory agents can be prescribed to the subject. Any of the diets, drugs, agents, or activities which can be prescribed to the patient can also be provided to the patient.

PRPC can be determined by a careful examination, typically with the aid of an instrument, device, or apparatus. The examination can be accomplished using an opthalmoscope, typically with indirect opthalmoscopy of the peripheral retina. The peripheral retina can also be photographed, most easily using wide-field systems, although experienced photographers may be able to capture images with standard systems. Such cameras include fundus or retinal cameras. Cameras may make and/or store images on a digital or film medium, for example. Images, particularly digital images, may be transformed or enhanced using algorithms, for example, to enhance contrast. Images may be analyzed electronically or by a human. The peripheral (at or anterior to equator) retinal pigment epithelial changes can be graded. In one grading system that we have developed, Grade 1 is a linear pattern of hyperpigmented lines, which may have some branching. Grade 2 is a linear patter of hyperpigmented lines with formation of incomplete geometric patterns or polygons; moderate pigmentation is also observed. Grade 3 is hyperpigmented lines forming complete polygons, such as five or six-sided geometric patterns; more marked hyperpigmentation is observed. The grading can be accomplished by a human. All three grades constitute PRPC.

As is known in the medical arts and sciences, a single diagnostic or prognostic parameter may or may not be relied upon in isolation. A number of different parameters may be considered in combination, including but not limited to patient age, general health status, sex, lifelong health habits, medication history, and physical or clinical findings. The latter may include, macular or extramacular drusen, retinal pigment epithelial changes, subretinal fluid, subretinal hemorrhage, disciform scarring, subretinal exudate, peripheral drusen, and peripheral reticular pigmentary change.

The Y402H polymorphism of CFH is encoded by the T1277C polymorphism. The CFH gene and protein are both known in the art.⁶

When an increased risk of neovascular AMD is identified or an early onset of neovascular AMD is identified, patients can be grouped appropriately, i.e., stratified so that appropriate conclusions can be drawn in clinical studies. Additionally, appropriate modifications to lifestyle can be recommended, including, but not limited to diet, supplementation of vitamins and minerals, for example, smoking cessation, drugs, and obesity reduction or control. Supplementation of diet, including but not limited to vitamins C, E, beta carotene, zinc, and/or lutein/zeaxanthin may be recommended. Diets high in these factors may be used as a source of the helpful factors. One particular combination supplement includes: 500 milligrams of vitamin C, 400 milligrams of vitamin E, 15 milligrams of beta-carotene, 80 milligrams of zinc as zinc oxide, two milligrams of copper as cupric oxide. Drugs which may delay onset or reduce progression of disease when it occurs include anti-inflammatory medicaments. Many are known in the art and can be used. Positive dietary recommendations include carrots, corn, kiwi, pumpkin, yellow squash, zucchini squash, red grapes, green peas, cucumber, butternut squash, green bell pepper, celery, cantaloupe, sweet potatoes, dried apricots, tomato and tomato products, dark green leafy vegetables, spinach, kale, turnips, and collard greens. Prescriptions and recommendations can be delivered orally. Best practices, however, require recording in a permanent data storage medium such as in a paper medical history or in an electronic medical history. Assignments to groups for a clinical study will also be recorded on a permanent data storage medium.

Identifying a patient with a diagnosis or prognosis typically involves an act of communicating a result or conclusion based on data interpretation. The form of communication may be in writing, oral, or electronic. The communication may be to the patient or patient's family member or caregiver; to a medical record; to a doctor; to a pharmacist; to a nurse; to an insurer; or to a health maintenance organization. Even if communicated orally, best practices require recording in a permanent data storage medium, such as on a paper or electronic storage medium or medical file.

Complement factor H was the first major AMD risk gene described but its effect on specific phenotypes within AMD has not been explored in detail.⁶⁻⁸ Our results suggest that individuals with AMD who possess the CFH Y402H variant (C allele at rs1061170) more commonly have PRPC compared to those who do not possess this variant. Further, a dose response curve exists between the number of variant alleles and the proportion of patients demonstrating PRPC.

These data represent a single point in time for each patient. We did not emphasize the near significance of the difference in grade between eyes within a participant (P=0.0026). In AMD, there is a continuum of disease that typically occurs at different rates between eyes. Patients often present with decreased vision in one eye and the second eye may follow sometime later. Our study was not longitudinal and therefore we could not determine if this difference would ultimately reach statistical significance or not.

It is unknown whether macular RPE hyperpigmentation and the PRPC found in AMD share the same etiology. However, it is interesting that there was no statistically significant difference between the groups with regard to the presence or absence of macular RPE hyperpigmentation despite the significant difference in PRPC.

Extramacular phenotypic variations are ignored by present AMD classification schemes;¹³⁻¹⁵ however, these peripheral findings are clearly important. For example, peripheral drusen are the only significant phenotypic difference demonstrated between singleton and multiplex probands with stage three or worse AMD (P=0001).¹² Though inclusion of peripheral fundus findings into AMD classification schemes would require more effort and cost, it may be warranted in order to better subdivide the phenotypic continuum of AMD. A more specific grouping of individuals with AMD may be beneficial from both a clinical and research standpoint.

For example, phenotypic characteristics appreciated during routine examination that suggest a high-risk genotype could prove valuable to the clinician, especially if such features can be identified early in the disease course. Identifying high-risk characteristics could become increasingly important as preventative and therapeutic options increase for AMD. Further, signs of high-risk genotypes could have prognostic implications, allow better patient education, follow-up, and modification of other risk factors.

A single susceptibility gene does not account for all the signs of AMD. Instead, interactions between multiple genes, as well as dietary and environmental factors in the setting of the aging process all contribute to the phenotype. This may explain why, despite the statistically significant dose response curve evident between the Y402H variant and PRPC, nearly half of the cases with two C alleles at rs1061170 did not have PRPC. Additionally, it is unknown when PRPC initially presents so it is also possible that more cases may develop PRPC.

Improved characterization of the genotype-phenotype relationship in AMD may provide insights into the molecular mechanisms of different subtypes of AMD and make the investigation of the non-genetic components more straightforward. Because the macula may be more prone to environmental effects due to anatomic, optical, and vascular factors, peripheral changes may suggest a more global retinal dysfunction with a genetic etiology compared to central findings alone.¹⁸ Further if peripheral changes occur at a younger age, it may suggest a more “severe” diffuse phenotype than macular changes alone¹⁸ or a different disease mechanism. Because CFH is involved in regulating the complement cascade and there are well known signs of retinal hyperpigmentation secondary to ocular inflammation (perivascular hyperpigmentation secondary to retinal vasculitis, for example), one could hypothesize that these PRPC may be a sign of diffuse immune dysregulation in the retina.

While many questions remain, a further understanding of the genotype-phenotype relationship in AMD may improve therapeutic recommendations, provide more accurate diagnosis, and lead to better understanding of the mechanism of this complex disease.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### EXAMPLE 1

### Methods

Patients were identified in the clinic populations of the Duke University Eye Center and the Vanderbilt Eye Institute or from referrals to the study centers by local ophthalmologists. Information was collected and protected in compliance with Health Insurance Portability and Accountability Act regulations, Institutional Review Board (IRB) approval was obtained, and all patients provided informed consent.

The clinical criteria, grading methodology, and grades used to define AMD have been previously described.¹² Age-related findings including drusen, retinal pigment epithelial (RPE) changes, neovascularization, and geographic atrophy were used to diagnose AMD in individuals 55 years of age or older.

Data collection for each participant was performed using a standardized protocol. Age and gender were noted. Medical, ocular, and family ocular histories were obtained. Most study participants completed these questionnaires in person with the clinical study coordinator present. Height, weight, and blood pressure were measured during the clinical encounter. Additional questionnaires were used to obtain lifelong health habits such as smoking, dietary supplementation, and sunlight exposure as well as current dietary practices. Patients usually completed these questionnaires at home or less frequently over the phone. Each individual received an ophthalmic examination that included slit lamp examination, biomicroscopy with a handheld 90-diopter lens or fundus contact lens, and (20 diopter) indirect opthalmoscopy of the peripheral retina.

The study protocol included at least three standard fields of thirty-five millimeter color fundus photographs as well as stereo photographs of the disc and macula. Authors, Eric A. Postel and Anita Agarwal, used the previously described modified Age-Related Eye Disease Study (AREDS) grading system to grade macular findings.¹³⁻¹⁵ Grade was based on the more severely affected eye. If multiple grades were present within an eye, the more severe grade was applied. Each eye of every individual received a grade.

Photographs were evaluated using stereoscopic magnification and standardized illumination (6000 k). Whenever possible, the Lens Opacities Classification System III standards were utilized to grade lenses.¹⁶ Detailed information was recorded from clinical and photographic examination regarding the presence of extramacular (around the arcades) and peripheral (anterior to the equator) drusen, peripheral reticular pigmentary change, posterior vitreous detachment, and iris color.

Individuals with grade 3 (Table 1) or higher disease in at least one eye were considered affected. Reliability of grading has previously been examined and concordance was found in 92%, with a Kappa score of 0.81.12 The power to detect a difference of 0.5 or more grade units between photos evaluated by each grader was over 99%.¹²

Phenotypic features investigated included: gender, age, AMD grade, difference in AMD grade, body mass index, Early Treatment Diabetic Retinopathy Study (ETDRS) visual acuity, refractive error, intraocular pressure, cataract assessment (presence or absence), nuclear color, nuclear opalescence, cortical lens opacity, posterior subcapsular cataract, iris color, RPE hyperpigmentation, RPE hypopigmentation, geographic atrophy, drusen (small, medium or large), extramacular drusen, pigmented epithelial detachment, subretinal fluid, subretinal hemorrhage, choroidal neovascular membrane (CNVM), disciform scarring, other signs of CNVM (e.g., subretinal exudates), laser scars, peripheral drusen, and peripheral reticular pigmentary change (PRPC). Other phenotypic ocular characteristics recorded included cup-to-disc ratio, glaucomatous cupping, optic atrophy, other optic disc conditions, and posterior vitreous detachment as evidenced by a Weiss ring.

The rs1061170 single nucleotide polymorphism (SNP) (C allele at rs1061170 or Y402H) was genotyped with a TaqMan assay using previously published probe sequences.¹⁷ We confirmed that genotypes generated by this assay agreed with genotypes generated by sequencing of this region.⁶

The presence of the CFH Y402H variant was tested for association with AMD affection status and other phenotypic characteristics. AMD cases possessing the Y402H variant (“Y402H carriers”) were independently analyzed for association with any of the phenotypic characteristics investigated and compared to AMD cases lacking this CFH variant (“Y402H non-carriers”). Statistical analyses were performed using SAS version 8.2 (SAS Institute, Cary, N.C., USA). Conservatively assuming all 34 comparisons are statistically independent, the Bonferroni correction required P≦0.001 to declare statistical significance. Phenotypic differences among groups were compared with a chi-square test for categorical variables. When the expected size of any cell in the resulting contingency table was below 5, Fisher's exact test was used instead. Continuously scaled variables were compared with Student's t-test. Dose-response relationships were evaluated by a Mantel-Haenszel test.

### EXAMPLE 2

### Results

The data set contained 956 unrelated AMD cases. Of these, the CFH Y402H variant was present in 796 carriers (344 homozygous, 452 heterozygous) and absent in 160 non-carriers. The demographics of Y402H carriers and non-carriers were statistically similar (Table 1). The mean age of the Y402H carrier group was 75.83 years versus the non-carrier group 75.84 years (P=0.980). The proportion of females in the carrier versus non-carrier group was 0.661 and 0.650, respectively (P=0.769). The proportion of grade 3, 4, and 5 AMD cases was similar between both groups (Table 1).

TABLE 1
Demographics of control and complement factor H (CFH) Y402H
polymorphism groups with age-related macular degeneration (AMD).

| | CFH | CFH | |
|---|---|---|---|
| | variant | variant | |
| | non-carrier | carrier | P |
| | group | group | value |
| Number of AMD cases | 160 | 796 | |
| Mean age (years) | 75.84 | 75.83 | 0.980 |
| Proportion of females | 0.650 | 0.661 | 0.769 |
| Proportion of grade 3 cases | 0.206 | 0.247 | 0.265 |
| Proportion of grade 4 cases | 0.125 | 0.144 | 0.519 |
| Proportion of grade 5 cases | 0.669 | 0.608 | 0.149 |
| Mean visual acuity | 188.4 | 178.3 | 0.738 |
| (ETDRS*) | | | |

*Early Treatment Diabetic Retinopathy Study (ETDRS)

We compared the CFH Y402H carrier and non-carrier groups regarding 34 phenotypic features (Table 2).

TABLE 2
Comparison of proportions of age-related macular degeneration (AMD)
cases with phenotypic features possessing increasing number of
complement factor H (CFH) risk C alleles.

| | Zero | One | Two | |
|---|---|---|---|---|
| Phenotypic Feature | allele | allele | alleles | P-value |
| Grade | | | | |
| 3 | 0.206 | 0.254 | 0.241 | 0.6885 |
| 4 | 0.125 | 0.141 | 0.148 | |
| 5 | 0.669 | 0.605 | 0.611 | |
| Difference in AMD Grade | | | | |
| 0 | 0.642 | 0.599 | 0.660 | 0.0026 |
| 1 | 0.126 | 0.161 | 0.122 | |
| >1 | 0.232 | 0.240 | 0.218 | |
| Iris color | | | | |
| blue or grey (BG) | 0.108 | 0.133 | 0.168 | 0.0904 |
| green or hazel (GH) | 0.169 | 0.213 | 0.216 | |
| light brown (LB) | 0.200 | 0.215 | 0.202 | |
| dark brown (DB) | 0.177 | 0.116 | 0.067 | |
| Other | 0.346 | 0.323 | 0.347 | |
| Mean Body Mass Index | 26.5 | 26.1 | 26.5 | 0.5521 |
| Mean Spherical Equivalent | 0.732 | 0.397 | 0.334 | 0.2567 |
| Mean Intraocular Pressure | 15.4 | 15.7 | 15.6 | 0.7729 |
| Cataract Assessment (present/ | 0.586 | 0.596 | 0.615 | 0.8929 |
| absent) | | | | |
| Mean Nuclear Color | 2.32 | 2.27 | 2.45 | 0.4316 |
| Mean Nuclear Opalescence | 2.31 | 2.19 | 2.43 | 0.2521 |
| Mean Cortial Cataract | 1.15 | 0.94 | 1.15 | 0.1645 |
| Mean Posterior Subcapsular | 0.402 | 0.484 | 0.484 | 0.7701 |
| Cataract | | | | |
| RPE hyperpigmentation | 0.574 | 0.579 | 0.635 | 0.3765 |
| RPE hypopigmentation | 0.206 | 0.211 | 0.200 | 0.9449 |
| Geographic Atrophy | 0.216 | 0.299 | 0.316 | 0.1286 |
| Drusen: Small | 0.566 | 0.526 | 0.565 | 0.5970 |
| Drusen: Intermediate | 0.656 | 0.605 | 0.649 | 0.4848 |
| Drusen: Large | 0.466 | 0.578 | 0.563 | 0.1370 |
| Extramacular Drusen | 0.165 | 0.090 | 0.085 | 0.0554 |
| Pigment Epithelial Detachment | 0.159 | 0.086 | 0.065 | 0.0164 |
| Subretinal Fluid | 0.304 | 0.195 | 0.208 | 0.0496 |
| Subretinal Hemorrhage | 0.319 | 0.213 | 0.230 | 0.0706 |
| Choroidal Neovascular Membrane | 0.261 | 0.228 | 0.217 | 0.6411 |
| (CNVM) | | | | |
| Disciform Scarring | 0.261 | 0.297 | 0.357 | 0.1130 |
| Other Signs of CNVM | 0.079 | 0.106 | 0.110 | 0.6399 |
| Laser Scar | 0.120 | 0.135 | 0.175 | 0.2650 |
| Peripheral Drusen | 0.101 | 0.125 | 0.145 | 0.5382 |
| Peripheral Reticular Pigment | 0.347 | 0.406 | 0.545 | 0.0006 |
| Changes | | | | |
| Mean Cup-to Disc Ratio | 0.282 | 0.348 | 0.334 | 0.2013 |
| Glaucomatous Cupping | 0.018 | 0.056 | 0.024 | 0.0700 |
| Optic Atrophy | 0.009 | 0.009 | 0.008 | 0.9876 |
| Other Optic Disc | 0.009 | 0.039 | 0.028 | 0.2545 |
| Posterior vitreous detachment | 0.327 | 0.332 | 0.307 | 0.8156 |

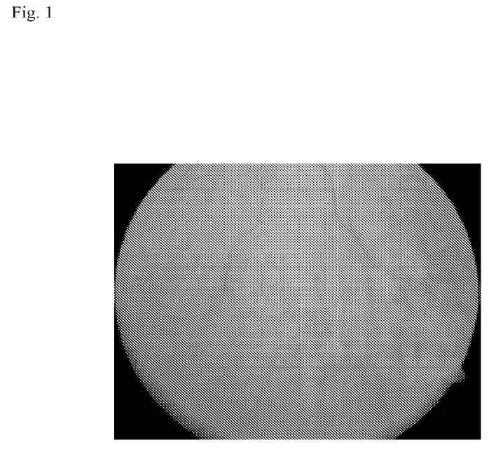

After Bonferroni correction requiring a P value less than 0.001, the only statistically significant difference between the Y402H carrier group versus the non-carrier group was the presence of PRPC (P=0.0006) (FIG. 1, Table 2). As the number of CFH risk (C at rs1061170, Y402H) alleles increased, the proportion of AMD cases with PRPC increased (Table 3). This dose-response correlation was statistically significant using the Mantel-Haenzel test (P=0.0002).

TABLE 3
Comparison of increasing number of complement factor H (CFH)
Y402H polymorphism alleles with the number and proportion of cases
demonstrating peripheral pigmentary change (PRPC).

| | Number | | Number | Proportion | |
|---|---|---|---|---|---|
| | of cases | Proportion of | of cases | of cases | |
| | without | cases | with | with | |
| | PRPC | without PRPC | PRPC | PRPC | Total |
| 0 CFH risk C | 64 | 0.653 | 34 | 0.347 | 98 |
| alleles | | | | | |
| 1 CFH risk C | 177 | 0.596 | 120 | 0.404 | 297 |
| alleles | | | | | |
| 2 CFH risk C | 102 | 0.455 | 122 | 0.5455 | 224 |
| alleles | | | | | |
| Total | 343 | | 276 | | 619 |

There was no statistically significant difference found between groups regarding any of the remaining features investigated requiring P≦0.001 (Table 2). However, the difference in AMD grade between eyes (P=0.0026) very nearly reached statistical significance.

### REFERENCES

The disclosure of each reference cited is expressly incorporated herein for the purpose to which is referenced in the text.
1. Tomany S C, Wang J J, Van Leeuwen R, Klein R, Mitchell P, et al. Risk factors for incident age-related macular degeneration: pooled findings from 3 continents. Opthalmol 2004; 111:1280-7.
2. Mitchell P, Smith W, Wang J J. Iris color, skin sun sensitivity, and age-related maculopathy. The Blue Mountains Eye Study. Opthalmol 1998; 105:1359-63.
3. Age-Related Eye Disease Study Research Group. Risk factors associated with age-related macular degeneration. A case-control study in the age-related eye disease study: Age-Related Eye Disease Study Report Number 3. Opthalmol 2000; 107:2224-32.
4. Lewis H, Straatsma B R, Foos R Y, Lightfoot D O. Reticular degeneration of the pigment epithelium. Opthalmol 1985; 92:1485-95.
5. Humphrey W T, Carlson R E, Valone J A Jr. Senile reticular pigmentary degeneration. Am J Opthalmol 1984; 98:717-22.
6. Haines J L, Hauser M A, Schmidt S, et al. Complement factor H variant increases the risk of age-related macular degeneration. Science 2005; 308:419-21.
7. Klein R J, Zeiss C, Chew E Y, et al. Complement factor H polymorphism in age-related macular degeneration. Science 2005; 308:385-9.
8. Edwards A O, Ritter R 3rd, Abel K J, et al. Complement factor H polymorphism and age-related macular degeneration. Science 2005; 308:421-4.
9. Magnusson K P, Duan S, Sigurdsson H, et al. CFH Y402H confers similar risk of soft drusen and both forms of advanced AMD. PLoS Med 2006; 3(1):e5 doi:10.1371/journal.pmed.0030005.
10. Sepp T, Khan J C, Thurlby D A, et al. Complement factor H variant Y402H is a major risk determinant for geographic atrophy and choroidal neovascularization in smokers and nonsmokers. Invest Opthalmol Vis Sci 2006; 47:536-40.
11. Postel E A, Agarwal A, Caldwell J, et al. Complement Factor H increases risk for atrophic age-related macular degeneration. Opthalmol 2006; 113(9):1504-7.
12. Postel E A, Agarwal A, Schmidt S, et al. Comparing age-related macular degeneration phenotype in probands from singleton and multiplex families. Am J Opthalmol 2005; 139:820-5.
13. De la Paz M A, Pericak-Vance M A, Haines J L, Seddon J M. Phenotypic heterogeneity in families with age-related macular degeneration. Am J Opthalmol 1997; 124:331-43.
14. Age-Related Eye Disease Study Research Group. The Age-Related Eye Disease Study system for classifying age-related macular degeneration from stereoscopic color fundus photographs: the Age-Related Eye Disease Study Report Number 6. Am J Opthalmol 2001; 132:668-81.
15. Age-Related Eye Disease Study Research Group. The Age-Related Eye Disease Study (AREDS): design implications. AREDS report no. 1. Control Clin Trials 1999; 20:573-600.
16. Chylack L T Jr, Wolfe J K, Singer D M, et al. The Lens Opacities Classification System III. The Longitudinal Study of Cataract Study Group. Arch Opthalmol 1993; 111:831-6.
17. Hageman G S, Anderson D H, Johnson L V, et al. A common haplotype in the complement regulatory gene factor H (HF1/CFH) predisposes individuals to age-related macular degeneration. Proc Natl Acad Sci USA 2005; 102:7227-32.
18. Traboulsi E I. The challenges and surprises of studying the genetics of age-related macular degeneration. Am J Opthalmol 2005; 139:908-11.
19. NCBI Reference Sequence NP_{—}00177 and NM_{—}000186

We claim:

1. A method of predicting increased risk of Age-Related Macular Degeneration, comprising:
examining a subject to determine the presence of peripheral reticular pigmentary change; and
predicting an increased risk of Age-Related Macular Degeneration when peripheral reticular pigmentary change is determined in the subject.

2. The method of claim 1 wherein the step of examining is instrument assisted.

3. The method of claim 1 wherein the step of examining comprises making an image of the peripheral retina.

4. The method of claim 1 wherein the step of examining employs an opthalmoscope.

5. The method of claim 1 wherein the step of examining employs indirect opthalmosocopy.

6. The method of claim 1 wherein the step of examining employs a camera.

7. The method of claim 1 wherein the step of examining employs a wide-field camera lens.

8. The method of claim 1 further comprising:
prescribing a dietary supplement to the subject to reduce and/or delay symptoms of Age-Related Macular Degeneration.

9. The method of claim 1 further comprising:
prescribing a diet to the subject to reduce and/or delay symptoms of Age-Related Macular Degeneration.

10. The method of claim 1 further comprising:
prescribing smoking cessation education and/or treatments for the subject to reduce and/or delay symptoms of Age-Related Macular Degeneration.

11. The method of claim 1 further comprising:
prescribing a monitoring schedule to the subject to monitor onset and/or progress of Age-Related Macular Degeneration.

12. The method of claim 1 further comprising:
assigning the subject to a clinical trial group with other subjects with peripheral reticular pigmentary change.

13. The method of claim 1 further comprising:
prescribing anti-oxidants to the subject to reduce and/or delay symptoms of Age-Related Macular Degeneration.

14. The method of claim 1 further comprising:
prescribing exercise to the subject to reduce and/or delay symptoms of Age-Related Macular Degeneration.

15. The method of claim 1 further comprising:
prescribing an anti-inflammatory agent to the subject to reduce and/or delay symptoms of Age-Related Macular Degeneration.

16. The method of claim 1 wherein the step of predicting involves recordation on a permanent data storage medium.

17. The method of claim 8, 9, 10, 11, 13, 14, or 15 wherein the step of prescribing employs recordation on a permanent data storage medium.

18. The method of claim 12 wherein the step of assigning employs recordation on a permanent data storage medium.
